(19) United States
(12) Patent Application Publication
(10) Pub. No.: US 2026/0248641 A1
(43) Pub. Date: Aug. 27, 2026

(54) Lip Condom

(71) Applicant: Shanstella Yvonne Barnes, Chicago, IL (US)

(72) Inventor: Shanstella Yvonne Barnes, Chicago, IL (US)

(73) Assignee: Buzz Guards Inc, Chicago, IL (US)

(21) Appl. No.: 19/064,658

(22) Filed: Feb. 26, 2025

(51) Int. Cl. A61F 6/04; A45D 44/12; A61F 6/06

(52) U.S. Cl. CPC A61F 6/04; A45D 44/12; A61F 6/065; A61F 2006/042; A61F 2006/047

(57) ABSTRACT

The lip Condom relates to the field of sexual health, protecting a user's lips from another person's lips as a practice of prevention during french kissing, intimacy, and/or intercourse, essentially acting as a barrier to protect a person's lips from the unknown contamination of another person. Most are aware of practicing “safe sex,” via using a penis condom, which the lack thereof can result in pregnancy, HIV, STIs, STDs, but not much emphasis is placed on covering the lips as a practice of prevention and protection. Thus, the combined usage of the traditional condom along with the lip condom, may be considered “safer” sex. The invention comprises one lip-shaped protective device, wherein the upper lip and lower lip contain more dense composite materials and wherein less material is configured to the extended parts on the left, upper, right, and lower sides; and adhesive.

### BACKGROUND OF THE INVENTION

The lip Condom relates to the field of sexual health, protecting a user's lips from another person's lips as a practice of prevention during french kissing, intimacy, and/or intercourse, essentially acting as a barrier to protect a person's lips from the unknown contamination of another person.

The device differs from what currently exists in function, i.e. lip moisturizers or healing agents, and differs in design i.e. patches or traditional condoms.

Most are aware of practicing “safe sex,” via using a penis condom, which the lack thereof can result in pregnancy, HIV, STIs, STDs, but not much emphasis is placed on covering the lips as a practice of prevention and protection. Thus, the combined usage of the traditional condom along with the lip condom, may be considered “safer” sex.

### SUMMARY OF THE INVENTION

Manufacturing may be accomplished by combining latex, elastomer, hydrocolloid and/or other durable, waterproof, elastic material, practical for sexual intimacy, enduring a reasonable amount of exposure without drying out. One skilled in the art of manufacturing and molding may realize other methods. The product is finished into the shape of an average person's full lips as one piece, with a more dense composite on the upper and lower lips, and less composite on the surrounding outer parts of the lips. The back side of the invention provides adhesive to aid in the device remaining in place, and the invention provides an opening wide and flexible enough to allow for french kissing without the need to be removed from the lips.

Color and/or artwork may be added using rubber stamps, silk screening, or other suitable methods. Flavored lubricant may be provided by adding glucose or glycerin (glycerol) to the lube, or other suitable methods.

### BRIEF DESCRIPTION OF THE DRAWING FIGURES

FIG. 1 shows a front, centered, perspective view of the device, covering the full lips as one piece, with a more dense composite on the upper and lower lips, and illustrates the lip device extended to the outer parts on the left, upper, right, and lower parts of the lips with a less dense composite.

FIG. 2 shows adhesive on the back side of the lip protective device and illustrates the peel away process.

### DESCRIPTION OF THE INVENTION

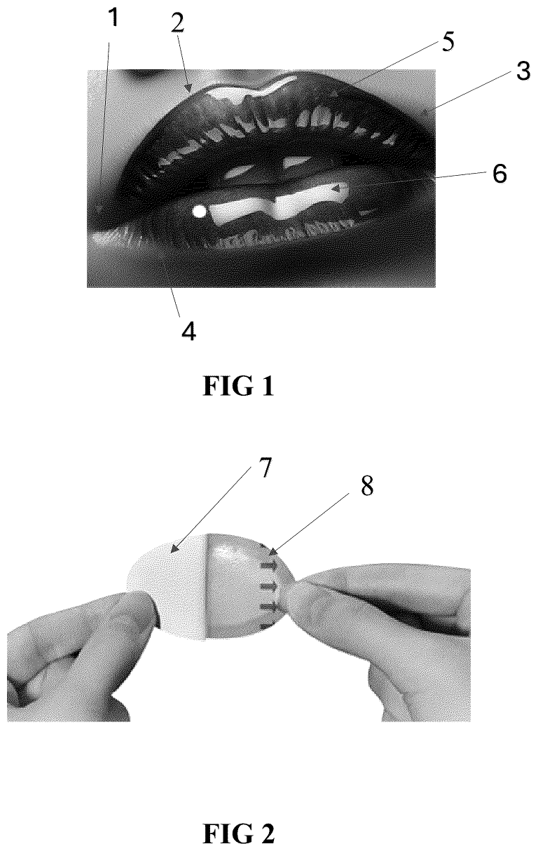

Looking at FIG. 1, it can be seen that the lip protective device covers the full set of lips as one lip-shaped piece, with less composite on the surrounding outer parts of the lips 1, 2, 3, and 4, and a more dense composite on the upper and lower lips 5, and 6, which completes the lip protection invention. FIG. 2 shows adhesive on a back side view 7, and illustrates the peel away process 8, of how the device is to be removed from the backing in order to be attached to the lips.

1. (canceled)

2. (canceled)

3. (canceled)

4. (canceled)

5. (canceled)

6. (canceled)

7. (canceled)

8. A lip shaped protective device to cover and protect a user's full lips from another person's lips due to french kissing and/or intimacy comprising:
a. one lip-shaped piece; and
b. wherein the device has a more dense composite on the upper and lower lips, and a less dense composite on the outer parts of the lips.

9. A lip shaped protective device to cover a user's full lips as one piece, if cleft lip, or other lip distortions may exist.
